(19) United States
(12) Patent Application Publication
(10) Pub. No.: US 2024/0226111 A1
(43) Pub. Date: Jul. 11, 2024

(54) COMPOSITION FOR TREATING OSTEOARTHRITIS

(71) Applicant: AJOU UNIVERSITY INDUSTRY-ACADEMIC COOPERATION FOUNDATION, Suwon-si (KR)

(72) Inventor: Siyoung Yang, Suwon-si (KR)

(73) Assignee: AJOU UNIVERSITY INDUSTRY-ACADEMIC COOPERATION FOUNDATION, Suwon-si (KR)

(21) Appl. No.: 18/440,990

(22) Filed: Feb. 14, 2024

Related U.S. Application Data
(63) Continuation of application No. PCT/KR2022/011975, filed on Aug. 11, 2022.

(30) Foreign Application Priority Data
Aug. 17, 2021 (KR) 10-2021-0107881
Aug. 17, 2021 (KR) 10-2021-0107882

(51) Int. Cl. A61K 31/551; A61P 19/02; C12N 15/113

(52) U.S. Cl. CPC A61K 31/551; A61P 19/02; C12N 15/113; C12N 2310/11; C12N 2310/12; C12N 2310/14; C12N 2310/141; C12N 2310/531

(57) ABSTRACT

The present disclosure relates to a composition for treating osteoarthritis or eliminating senescent cells, including K-7174 that targets zinc finger MIZ domain-containing protein 1 (Zmiz1) as an active ingredient, wherein suppression of overexpression of Zmiz1 found in osteoarthritis-induced joint tissues or mice as well as suppression of expression, due to binding between K-7174 and GATA whose expression is induced by the Zmiz1 overexpression, of joint destructing factors (Mmp3, Cox2) and senescence cell promoting factors (Glb1, p16) whose expressions are increased upon the overexpression have shown effects of decreasing the progression of osteoarthritis and promoting elimination of senescent cells, such that it is possible to provide a composition including the K-7174 as an active ingredient as a treatment for osteoarthritis or an agent for eliminating senescent cells.

### CROSS-REFERENCE TO RELATED APPLICATION

This application is a continuation of PCT International Patent Application No. PCT/KR2022/011975 filed Aug. 11, 2022, which claims priority to Korean Patent Application Nos. 10-2021-0107881 filed on Aug. 17, 2021 and 10-2021-0107882 filed on Aug. 17, 2021 which are all hereby incorporated by reference in their entirety.

### BACKGROUND OF THE INVENTION

Osteoarthritis (OA) is a degenerative joint disease that is mainly caused by inhibition of cartilage ECM synthesis and promotion of cartilage destruction. Many etiological risk factors and pathophysiological processes related to aging contribute to the progression of osteoarthritis. Mechanical stress, including joint instability and damage, and age-related factors that make vulnerable to osteoarthritis are potential mechanisms causing osteoarthritis. Due to activated biochemical pathways in the chondrocyte, a unique cell type that produces a variety of catabolic and anabolic factors, these factors lead to degradation of the extracellular matrix (ECM) by matrix metalloproteinase (MMP) and disruption of ECM synthesis through dedifferentiation and apoptosis of chondrocytes. Especially, once the cartilage tissue that makes up the joints is damaged, it cannot be regenerated properly in the body. When such the cartilage tissue in the joint is damaged, severe pain occurs to limit daily activities, and when becoming chronic, it may lead to fatal osteoarthritis and interfere with normal life and professional activities.

On the other hand, in the case of rheumatoid arthritis (RA), unlike osteoarthritis caused by destruction of chondrocytes and cartilage tissues, progression of the disease due to an autoimmune response is known to be an important causative factor. Rheumatoid arthritis is a chronic autoimmune disease characterized by inflammation and proliferation of synovial cells, and unlike osteoarthritis, osteoporosis and bone erosion in the bones around the joints occur. In the rheumatoid arthritis, inflammation in the synovial membrane spreads to joint capsules, ligaments, and tendons and then invades the bone. Therefore, osteoarthritis and rheumatoid arthritis are completely different in terms of causes and progression, and their treatment methods are also different.

Therapeutic agents for rheumatoid arthritis known to date include nonsteroidal anti-inflammatory drugs (NSAIDs), penicillamine, steroid hormones, TNF inhibitors, interleukin inhibitors, JAK inhibitors, and anti-CD-related inhibitors that are suitable to block inflammatory mechanisms. Although NSAIDs and steroid hormones are being used in patients with osteoarthritis to relieve joint pain and inflammation, they are not practical treatment for osteoarthritis since they only relieve symptoms rather than treat the disease itself. Moreover, osteoarthritis, which is mainly caused by destruction of chondrocytes and cartilage tissues, is quite different in terms of causes and symptoms of the disease from rheumatoid arthritis, which is inflammatory arthritis, such that treatment methods for osteoarthritis should be different from that of rheumatoid arthritis as well.

Therefore, early diagnosis of osteoarthritis is necessary to prevent or slow the progression of osteoarthritis, but so far, methods such as hospital examination or X-ray by orthopedics are only available to diagnose osteoarthritis.

Senescent cells have been found to play a crucial role in the development and progression of age-related diseases and disorders as the cells are accumulated in tissues and organs adjacent to age-related lesions during the aging process. In mouse models where genetic or pharmacological approaches are applied, elimination of senescent cells has revealed to prolong a healthy period while preventing or delaying occurrence of senescence-associated diseases and disorders as well as frailty. Since then, a number of pharmaceutical compounds that are able to selectively eliminate senescent cells have been identified, commonly referred to as “senolytic.”

When it comes to cellular senescence, it is believed that chronic accumulation of senescent cells in organs and tissues during the aging process is a major driving factor in the development and progression of age-related diseases and disorder. Senescent cells build up with age-related β-galactosidase (SA-β-gal) activity and exhibit typical morphology (Campisi and d'Adda di Fagagna 2007). Being chronically present, these cells express proteins (p16, Glb1) called senescence-associated secretory phenotypes (SASPs), which adversely affect surrounding tissues (Acosta et al. 2013; Copp et al. 2010; Krtolica et al. 2001).

### SUMMARY OF THE INVENTION

An object of the present disclosure is to provide a composition for treating osteoarthritis, including an expression or activity inhibitor of Zmiz1 as an active ingredient, by determining relationship between osteoarthritis and changes in expression or activity levels of ZMIZ1.

In addition, another object of the present disclosure is to provide a composition including K-7174 as an active ingredient as a composition for treating osteoarthritis and a senolytic composition.

### Technical Solutions

The present disclosure provides a pharmaceutical composition for treating osteoarthritis, including an expression or activity inhibitor of zinc finger MIZ domain-containing protein 1 (Zmiz1) as an active ingredient.

The present disclosure provides a pharmaceutical composition for treating an osteoarthritis disease, including K-7174 as an active ingredient.

In addition, the present disclosure provides a pharmaceutical composition for eliminating senescent cells, including K-7174 as an active ingredient.

According to the present disclosure, an increase in expression of Zmiz1 was observed in osteoarthritis-induced joint tissues, and at the same time, an increase in the joint destructing factors Mmp3 and Cox2, and senescent cell promoting factors Gl1b and p16 were observed. Due to binding of GATA whose expression is induced by overexpression of the Zmiz1 and K-7174, effects were identified that collagenase activity, expression of Mmp3 and Cox2 which are inflammation inducing factors, and expression of Glb1 and p16 which are senescent cell promoting factors were suppressed, and progression of osteoarthritis was inhibited, such that a composition including the K-7174 as an active ingredient may be provided as a treatment for osteoarthritis and an agent for eliminating senescent cells.

### BRIEF DESCRIPTION OF DRAWINGS

FIG. 1 shows results of identifying that ZMIZ1, MMP3, and COX2 are expressed in patients with osteoarthritis and joint tissues of osteoarthritis-induced mice.

FIG. 2 shows results of identifying an increase in expression of ZMIZ1, senescent cell promoting factors (GLB1, p16), and joint destructing factors (Mmp3, Cox2) in aged mice.

FIGS. 3A-3F show results of identifying that Mmp3 and Cox2 which induce joint destruction and senescent cell promoting factors Glb1 and p16 are increased, as a result of inducing osteoarthritis through destabilization of medial meniscus (DMM) after overexpression of Zmiz1 only in joints using Col2a1-Zmiz1 TG mice in which Zmiz1 is expressed joint-specifically. FIG. 3A shows results of a study that analyzed Col2a1-Zmiz1 TG mice and wild type (WT) mice through safranin-O staining to prove that Col2a1-Zmiz1 TG mice are prone to joint destruction, and FIG. 3B shows results of measuring a degree of joint destruction by measuring a degree of cartilage destruction (OARSI grade) and subchondral bone (SBP) thickness in FIG. 3A. FIGS. 3C and 3D show results of measuring expression of Zmiz1, Mmp3, and Cox2 through immunohistochemistry (IHC), and FIGS. 3E and 3F show results of identifying that senescent cell promoting factors Glb1 and p16 increase in Col2a1-Zmiz1 TG mice.

FIGS. 4A-4F show results of inhibiting cartilage destruction and eliminating senescent cells in the knee joint of mice injected with Ad-C and Ad-shZmiz1 into the joint, wherein FIG. 4A shows results of safranin-O staining in that cartilage destruction is inhibited when Zmiz1 expression is suppressed, and FIG. 4B shows results of measuring a degree of joint destruction by measuring a degree of cartilage destruction and SBP thickness in FIG. 4A. FIG. 4C shows results of immunohistochemistry (IHC) analysis to identify an increase in expression of Zmiz1, Mmp3, and Cox2, and FIG. 4D shows a result of measuring an expression level of Zmiz1, Mmp3, and Cox2 expressed in FIG. 4C via IHC % Area. FIG. 4E shows results of analyzing by immunohistochemistry (IHC) that expression of senescent cell promoting factors Glb1 and p16 is reduced by Ad-shZmiz1, and FIG. 4F shows a result of measuring an expression level of Glb1 and p16 expressed in FIG. 4E by IHC % Area.

FIGS. 5A-5F show results of identifying transcription factors regulated by Zmiz1 and analyzing that activity of Zmiz1 may be regulated by K-7174 by inhibiting the binding between Zmiz1 and transcription factors, wherein FIG. 5A shows a result of analyzing, through Dual-Glo Luciferase Assay system (Promega), transcription factors regulated by Zmiz1 by infecting chondrocytes with Ad-Zmiz1 to find transcription factors regulated by Zmiz1, and then, in order to analyze the transcription factors regulated by Zmiz1, transfecting chondrocytes with Cignal 45-Pathway Reporter (Qiagen, hilden, Germany) that primarily enables identification of 45 transcription factors, followed by infection with Ad-Zmiz1 for 36 hours, and FIGS. 5B and 5C show modeling results to determine how K-7174 inhibits the activity of Zmiz1 as Zmiz1 and GATA or Zmiz1 and K-7174 bind to each other. FIGS. 5D and 5E show results of determining that decreasing sulfate proteoglycans and increasing senescent cell promoting factors are reduced by K-7174 treatment when Zmiz1 is overexpressed in mouse joints cultured ex vivo, and FIG. 5F show result of identifying that the senescent cell promoting factors Glb1 and p16, which were increased by Zmiz1 overexpression, were reduced by K-7174.

FIGS. 6A-6E show results of determining that joint destruction, joint destructing factors and senescent cell promoting factors were inhibited by orally administrating K-7174 to osteoarthritis-induced animal models, wherein FIG. 6A is a schematic diagram showing a time and concentration of K-7174 administered after inducing osteoarthritis by DMM, FIG. 6B shows results of identifying through safranin-O staining that joints are not damaged by orally administering K-7174 (10, 25, and 50 mg/kg) every two days for 6 weeks in the manner shown in FIG. 6A and treating K-7174 by concentration (10, 25, and 50 mg/kg) to mice with DMM-induced degenerative arthritis, and at the same time based on these results, FIG. 6C shows results of numerically determining that K-7174 may suppress osteoarthritis and promote elimination of senescent cells by measuring the OARSI grade and SBP thickness separately. FIG. 6D shows results of determining through immunohistochemistry analysis that expression of cartilage destructing factors (Mmp3, Cox2) and senescence cell promoting factors (Glb1, p16) are also reduced by K-7174 administration, and FIG. 6E shows results of measuring expression levels of Mmp3, Cox2, Glb1, and p16 per joint area by quantifying in accordance with FIG. 6D.

FIG. 7 is a diagram schematically showing a vector map required to prepare a ZMIZ1 gene-engineered mouse.

FIG. 8 is a schematic diagram showing a molecular mechanism by which K-7174 controls osteoarthritis through elimination of senescent cells.

### DETAILED DESCRIPTION OF THE INVENTION

Hereinafter, the present disclosure will be described in detail.

The inventors of the present disclosure have completed the present disclosure to enhance a therapeutic effect on osteoarthritis by regulating expression or activity of ZMIZ1 by determining an increase in the expression and activity of ZMIZ1 in osteoarthritis and consequently an increase in the expression of MMP3 and COX2, which induce cartilage destruction.

In addition, the inventors of the present disclosure have completed the present disclosure by identifying that the progression of osteoarthritis is inhibited, by suppressing collagenase activity, expression of Mmp3 and Cox2, which are inflammation inducing factors, and expression of Glb1 and p16, which are senescent cell promoting factors, as K-7174 binds to GATA whose expression is induced by Zmiz1 overexpressed in osteoarthritis-induced joint tissues.

The present disclosure may provide a pharmaceutical composition for treating osteoarthritis, including an expression or activity inhibitor of zinc finger MIZ domain-containing protein 1 (Zmiz1) as an active ingredient.

The zinc finger MIZ domain-containing protein 1 (Zmiz1) may have NCBI accession number. 57178 (Gene ID).

The expression inhibitor of Zmiz1 may be one or more selected from the group consisting of antisense nucleotides, siRNA, shRNA, microRNA, and ribozymes that bind specifically to mRNA of a Zmiz1 gene or a gene that promotes expression of Zmiz1.

The activity inhibitor of Zmiz1 may be one or more selected from the group consisting of compounds, peptides, peptidomimetics, proteins, aptamers, and antibodies that bind specifically to a protein that promotes Zmiz1 activity or a Zmiz1 protein.

The present disclosure may provide a pharmaceutical composition for treating an osteoarthritis disease, including K-7174 as an active ingredient.

As used herein, “K-7174” may include a compound represented by the following Chemical Formula 1 or a pharmaceutically acceptable salt thereof.

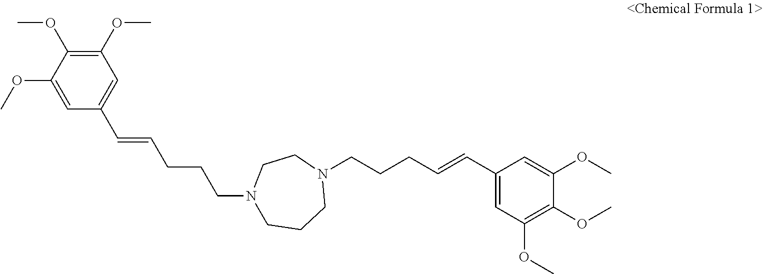

The compound may be named 1,4-bis((E)-5-(3,4,5-trimethoxyphenyl) pent-4-en-1-yl)-1,4-diazepane.

The compound may be provided in the form of a pharmaceutically acceptable salt within a range that it has the same efficacy.

As used herein, the term “pharmaceutically acceptable” refers to a salt that is not toxic to cells or humans exposed to the composition and secures safety and efficacy profiles suitable for administration to humans.

The salt may be used in either a pharmaceutically acceptable basic salt or acidic salt. The basic salt may be used in any one form of organic or inorganic basic salts and selected from the group consisting of sodium salts, potassium salts, calcium salts, lithium salts, magnesium salts, cesium salts, aminium salt, ammonium salts, triethylaminium salts, and pyridinium salts.

For acidic salts, acid additive salt that is formed by free acid may be useful. Inorganic acids and organic acids may be used as free acids, wherein hydrochloric acid, bromic acid, sulfuric acid, sulfurous acid, phosphoric acid, diphosphoric acid, and nitric acid may be used as the inorganic acid, and citric acid, acetic acid, maleic acid, malic acid, fumaric acid, gluconic acid, methanesulfonic acid, benzenesulfonic acid, camphorsulfonic acid, oxalic acid, malonic acid, glutaric acid, acetic acid, glycolic acid, succinic acid, tartaric acid, 4-toluenesulfonic acid, galacturonic acid, embonic acid, glutamic acid, citric acid, aspartic acid, and stearic acid may be used as the organic acid, but they are not limited thereby and may include all salts formed with various inorganic acids and organic acids that are commonly used in the art.

In addition, the compound may include not only the salt described above, but also all salts, hydrates, solvates, and derivatives which may be prepared by conventional methods. Additive salts may be prepared via conventional methods, by dissolving in a water-miscible organic solvent, such as acetone, methanol, ethanol, or acetonitrile and then adding an excess amount of organic base or by adding an aqueous basic solution of an inorganic base and then performing precipitation or crystallization. Alternatively, additive salts may be obtained by drying after evaporating the solvent or excess bases from the mixture or by performing aspiration and filtration of precipitated salts.

Preferably, it may include, but is not limited to, salts of hydrochloric acid thereof.

The K-7174 may bind to GATA, whose expression is induced by Zmiz1.

The K-7174 may suppress expression of joint destructing factors (MMP3 and COX2) and that of senescent cell promoting factors (Glb1, p16) induced by Zmiz1.

In other words, the K-7174 may have senolytic activity to selectively eliminate senescent cells.

In addition, the present disclosure provides a pharmaceutical composition for eliminating senescent cells, including K-7174 as an active ingredient.

The pharmaceutical composition may include the K-7174 in an amount of 0.1 to 10 parts by weight with respect to the total of 100 parts by weight of the composition.

In an example embodiment of the present disclosure, the pharmaceutical composition may use any one formulation selected from the group consisting of injections, granules, acids, tablets, pills, capsules, suppositories, gels, suspensions, emulsions, drips, or liquids in accordance with the conventional method.

In another example embodiment of the present disclosure, the pharmaceutical composition may additionally include one or more additives selected from the group consisting of appropriate carriers, excipients, disintegrators, sweeteners, coating agents, blowing agents, glydents, flavoring agents, antioxidants, buffers, bacteriostatic agents, diluents, dispersing agents, surfactants, binders, and lubricants that are commonly used in the preparation of the pharmaceutical composition.

Specifically for carriers, excipients, and diluents, lactose, dextrose, sucrose, sorbitol, mannitol, xylitol, erythritol, maltitol, starch, gum acacia, alginate, gelatin, calcium phosphate, calcium silicate, cellulose, methylcellulose, microcrystalline cellulose, polyvinyl pyrrolidone, water, methyl hydroxybenzoate, propyl hydroxybenzoate, talc, magnesium stearate and mineral oil may be used, and solid preparations for oral administration include tablets, pills, acids, granules, and capsules, wherein these solid preparations may be prepared by mixing at least one or more excipients in the composition, such as starch, calcium carbonate, sucrose or lactose, and gelatin. In addition to simple excipients, lubricants such as magnesium stearate and talc may also be used. Liquid preparations for oral administration may include suspensions, solutions, emulsions, and syrups, and also include various excipients, such as wetting agents, sweeteners, fragrances, and preservatives, in addition to the simple diluents that are commonly used, such as water and liquid paraffin. Preparations for parenteral administration include sterile aqueous solutions, non-aqueous solvents, suspensions, emulsions, lyophilized agents, and suppositories. As for non-aqueous solvents and suspensions, propylene glycol, polyethylene glycol, vegetable oils such as olive oil, and injectable esters such as ethyl oleate may be used. Witepsol, macrogol, tween 61, cacao butter, laurin fat, and glycerogelatin may be used as base materials for suppositories.

According to an example embodiment of the present disclosure, the pharmaceutical composition may be administered to a subject via a conventional method through intravenous, intraarterial, intraperitoneal, intramuscular, intrasternal, percutaneous, nasal, inhaled, topical, rectal, oral, intraocular, or intradermal routes.

The preferred dosage of the active ingredient such as the expression or activity inhibitor of Zmiz1 and K-7174 may depend on the condition and weight of a subject, the type and severity of a disease, a drug form, the administration route, and duration, and may be appropriately selected by the person skilled in the art. According to an example embodiment of the present disclosure, a daily dose may be, but is not limited to, 0.01 to 200 mg/kg, specifically 0.1 to 200 mg/kg, and more specifically 0.1 to 100 mg/kg. The administration may be conducted once a day or in several divided doses, but the scope of the present disclosure is not limited thereby.

In the present disclosure, the ‘subject’ may be a mammal including a human, but is not limited to these examples.

Hereinafter, the present disclosure will be described in detail through example embodiments to help understanding of the present disclosure. However, the following example embodiments are merely illustrative of the content of the present disclosure and the scope of the present disclosure is not limited to the following example embodiments. The example embodiments of the present disclosure are provided to more completely describe the present disclosure to those skilled in the art.

### EXPERIMENTAL EXAMPLE

The following experimental examples are intended to provide experimental examples that are commonly applied to each example embodiment according to the present disclosure.

### 1. Preparation of Human Osteoarthritis Samples and Experimental Osteoarthritis Mouse Models

Human cartilage samples were obtained from individuals aged 63 to 80 years who underwent total knee arthroplasty (Table 1). All patients submitted written informed consent, and sample collection was approved by the IRB from Catholic University (UC14CNSI0150).

Male C57BL/6 and Col2a1-Zmiz1 TG mice were kept in accordance with the guidelines of the Institutional Animal Care and Use Committee, which approved all animal procedures at the Center for Laboratory Animal Research in Ajou University.

TABLE 1
Characteristics of OA patients from
whom specimens were obtained.

| | | ICRS^{a} | | Weight | Height | |
|---|---|---|---|---|---|---|
| No. | Age/gender | grade | Joint | (kg) | (m) | BMI^{b(kg/m}^{²}⁾ |
| 1 | 65/F | 4 | Knee | 158 | 53 | 21.23 |
| 2 | 80/F | 4 | Knee | 143.3 | 55.1 | 26.83 |
| 3 | 72/F | 4 | Knee | 165 | 65 | 23.88 |
| 4 | 63/F | 4 | Knee | 152 | 52 | 22.51 |
| 5 | 69/F | 4 | Knee | 151 | 60 | 26.31 |
| 6 | 73/F | 4 | Knee | 153.8 | 70.75 | 29.89 |
| 7 | 63/F | 4 | Knee | 156 | 72.2 | 29.67 |
| 8 | 73/F | 4 | Knee | 154 | 83 | 35 |
| 9 | 75/F | 4 | Knee | 154 | 55 | 23.19 |
| 10 | 63/F | 4 | Knee | 163 | 74.3 | 27.96 |

^{a}ICRS, International Cartilage Repair Society;
^{b}BMI, Body Mass Index

To produce a model with osteoarthritis, 12-week-old male mice were subjected to surgical destabilization of the medial meniscus (DMM) and sacrificed at 10 weeks postoperatively. Female mice were excluded due to the effect of female hormones on pathogenesis of osteoarthritis. Adenoviruses for intra-articular injection were purchased from Vector Biolabs (Malvern, USA): Ad-C (1060), Ad-Zmiz1 (ADV-277084) and Ad-shZmiz1 (shADV-228281). Wild-type mice were injected with adenovirus Ad-shZmiz1 (1×10⁹ PFUs/10 μL) into the knee joint once weekly from 4 weeks after DMM surgery and sacrificed 7 weeks after the first adenovirus injection.

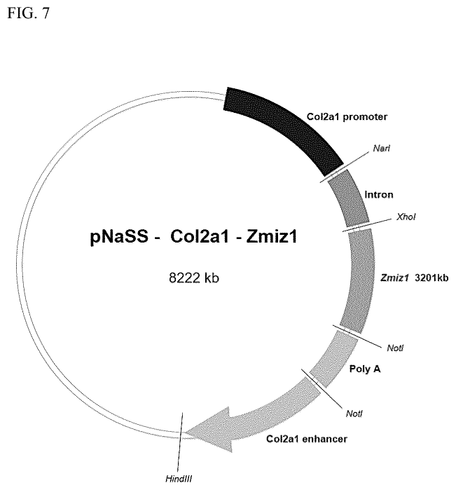

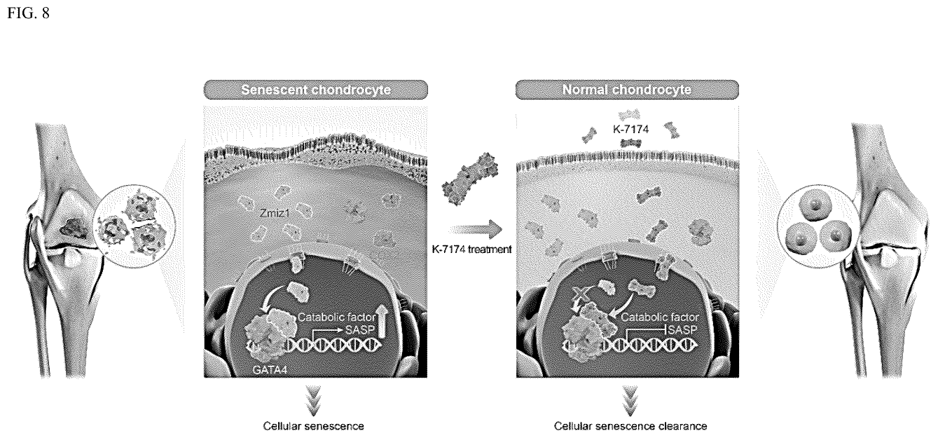

In the case of Col2a1-Zmiz1 TG mice, in order to prepare transgenic mice that chondrocyte-specifically overexpress Zmiz1, a transplant gene was prepared first (FIG. 7).

A fragment (3.201 kb) cleaved by NotI and NotI restriction enzymes from the Zmiz1 gene in mice was cloned to the NotI site of a pNass beta vector (Clontech Laboratories, USA) with a size of 4.35 kb. In addition, cloned were an enhancer and a promoter of collagen type II [CII, (Col2a1)] with a size of 1.0 kb that induce chondrocyte-specific expression at the NotI loci of the vector as well as an intron portion corresponding to 0.7 kb. The vector was cleaved with the NotI enzyme to obtain a purified transplant gene with a total size of 10.251 kb, which was named CII-pNass-ß-Zmiz1 construct (FIG. 7; gene 3.201 kb+vector 4.35 kb+enhancer 1.0 kb+promoter 1.0 kb+intron 0.7 kb=10.251 kb). The transplant gene was microinjected into the pronucleus of fertilized eggs of C57BL/6 mice (Charles River, Japan) to obtain a Col2a1-Zmiz1 TG mouse.

### 2. Culture of Mouse Joints

For Ex vivo culture, mouse cartilage was separated from the cartilage of ICR mice on day 5 after birth, enzymatically degraded with protease and collagenase, and then kept in DMEM (Capricorn science GmbH; Hessen, Germany) supplemented with 10% FBS, 100 units/mL of penicillin, and 100 μg/mL of streptomycin. It was infected with adenovirus or treated with K-7174.

### 3. Reagents

Antibodies of Zmiz1, COX2, MMP3, Glb1, and p16 were purchased from Abcam (Cambridge, UK), and K-7174 was purchased from Cayman (Michigan, USA; 32773).

### 4. Western Blotting

Chondrocytes were dissolved in a lysis buffer including 50 mM Tris-HCl (pH 7.5), 150 mM NaCl, 50 mM NaF, 1% Tween 20, 0.2% NP-40, and a protease inhibitor. The same amount of cell extracts was separated by SDS-PAGE (6% stacking gel and 10% running gel) and analyzed by immunoblotting.

### 5. Histology and Immunohistochemistry

Human osteoarthritis-induced cartilage and mouse knee joint were fixed in 4% paraformaldehyde and embedded in paraffin. The mouse knee joint was calcified in 0.5 M EDTA (pH 7.4) for 2 weeks. Paraffin-embedded samples were stained with safranin-O or alcian blue or immunostaining. Cartilage destruction was evaluated in an experimental osteoarthritis mouse model by three observers who were unaware of experimental grouping and scored according to the OARSI (Osteoarthritis Research Society International) grading system (grade 0-6). OARSI scores were presented as the mean maximum score for each mouse. Representative safranin-O staining images were selected from the highest advanced lesions in each section, and subchondral bone sclerosis was determined by measuring a thickness of subchondral bone. Immunohistochemical staining was performed on human and mouse cartilage sections using MMP3, COX2, Gl1b, p16, and Zmiz1 antibodies.

### 6. Analysis of Transcription Factors

Chondrocytes were primarily transfected with a Cignal 45-Pathway reporter (Qiagen, hilden, Germany) that enables identification of 45 transcription factors, and then infected with Ad-Zmiz1 for 36 hours to analyze the transcription factor regulated by Zmiz1 through the Dual-Glo Luciferase Assay System (Promega).

### 7. Discovery Studio Analysis

Discovery studio predicted binding sites to which GATA and K-7174 may bind.

### 8. Statistical Analysis

All experiments were conducted independently four or more times. Two independent groups were compared using the Shapiro-Wilk normality test, Levene's homogeneity of variance test, and the two-tailed independent t-test. Multiple comparisons were made using the Shapiro-Wilk test, Levene's test, and one-way analysis of variance with Bonferroni's post-hoc test. Non-parametric Mann-Whitney U tests were used to analyze data based on an ordinal grading system. P value of less than 0.05 was considered statistically significant.

### <Example 1> Identification of Association Between Changes in a Gene Expression Pattern by Overexpression of Zmiz1 and Osteoarthritis

To investigate a possible role of zinc finger MIZ domain-containing protein 1 (Zmiz1) in bone joints, Zmiz1 expression was identified in human and mouse osteoarthritis non-damaged and damaged cartilage samples.

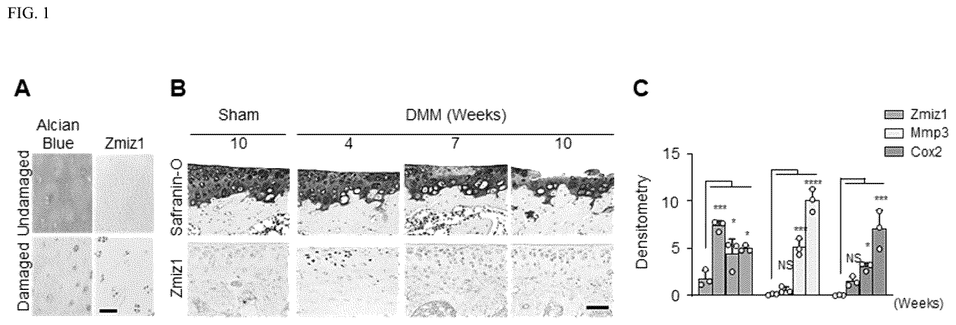

As a result, it was found that Zmiz1 expression was significantly higher in damaged cartilage with osteoarthritis than in non-damaged samples as shown in FIG. 1 (FIG. 1A; human, 1B; mouse). In particular, when osteoarthritis was induced in mice, it was found that Zmiz1 was expressed earlier than joint destructing factors (Mmp3, Cox2) (FIG. 1C).

In addition, since osteoarthritis is a representative geriatric disease, the expression pattern of ZMIZ1 was observed in aged mice.

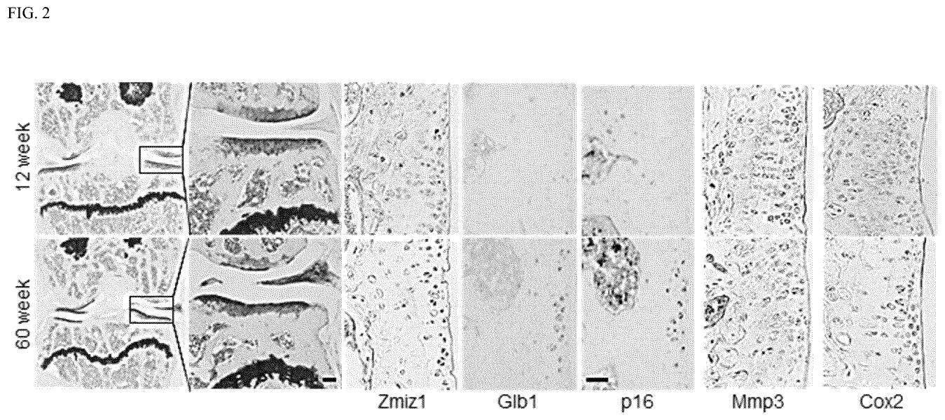

As a result, as shown in FIG. 2, it was found that the expression of senescent cell promoting factors Glb1 and p16 and joint destructing factors Mmp3 and Cox2 was increased in the aged mice, and at the same time, the expression of Zmiz1 was also increased.

### <Example 2> Identification of Onset of Osteoarthritis and Regulation of Senescent Cell Promotion by Zmiz1

To determine association between elevated Zmiz1 expression and pathogenesis of osteoarthritis and promotion of senescent cells that were found in the previous experiment, Col2a1-Zmiz1 TG mice in which Zmiz1 is joint-specifically overexpressed were analyzed.

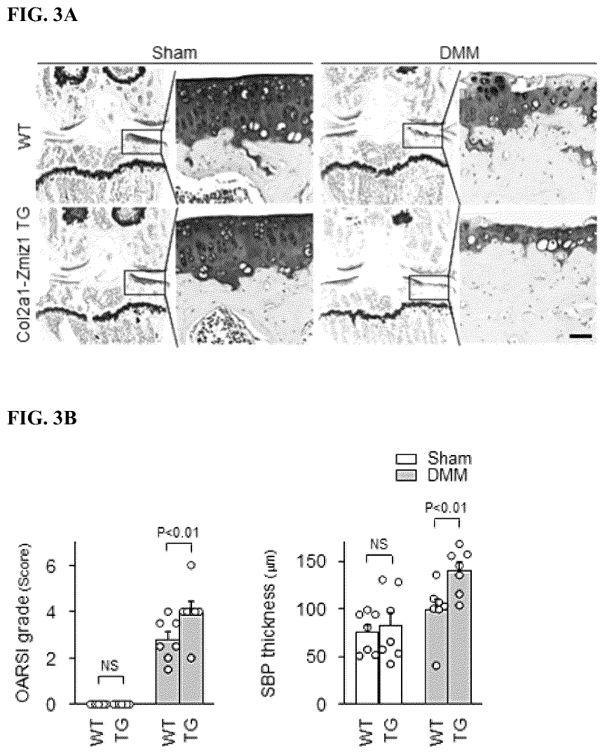

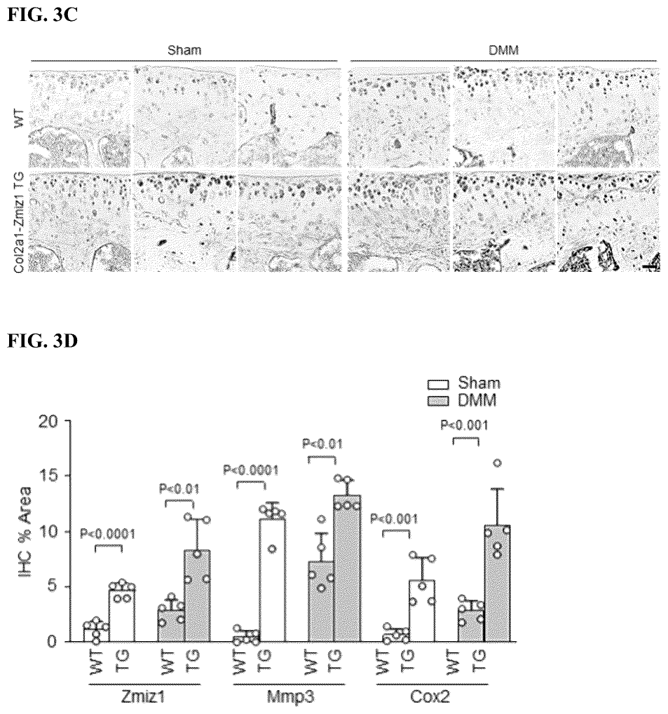

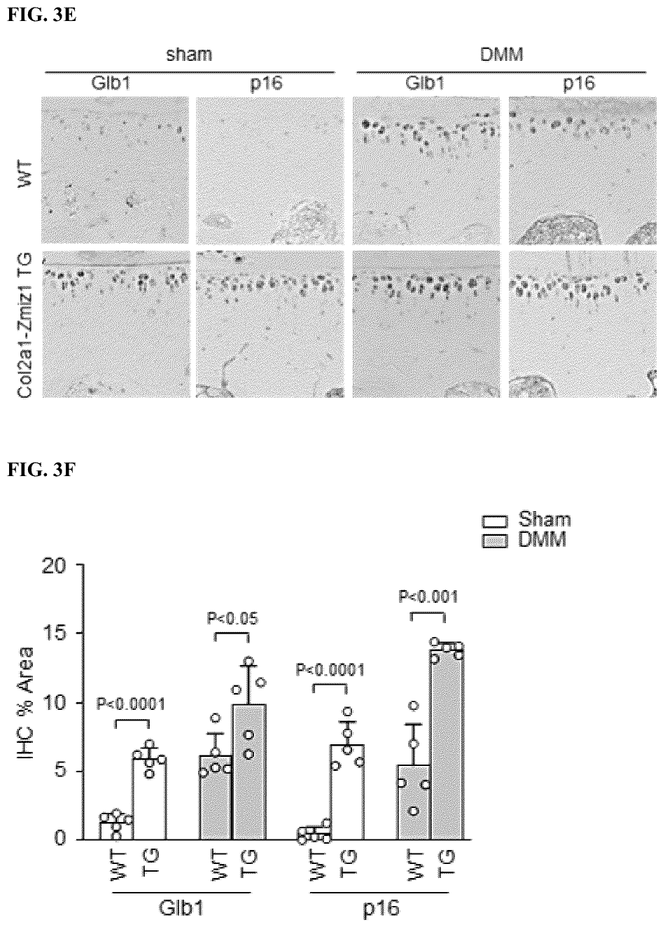

As a result, it was found that joint destruction was promoted in the Col2a1-Zmiz1 TG mouse as shown in FIGS. 3A and 3B, through safranin-O staining, OARSI grade, and SBP thickness. In addition, as shown in FIGS. 3C and 3D, joint destructing factors (MMP3, and COX2) were found to be overexpressed in the Col2a1-Zmiz1 TG mice. In addition, it was observed that Glb1 and p16, which are senescent cell promoting factors, increased as shown in FIGS. 3E and 3F.

### <Example 3> Identification of Regulation of Osteoarthritis by Inhibition of Zmiz1 In Vivo

Ad-shZmiz1 and Ad-C were injected into the knee by intra-articular injection using the adenovirus delivery system, and safranin-O staining and immunohistochemistry were performed.

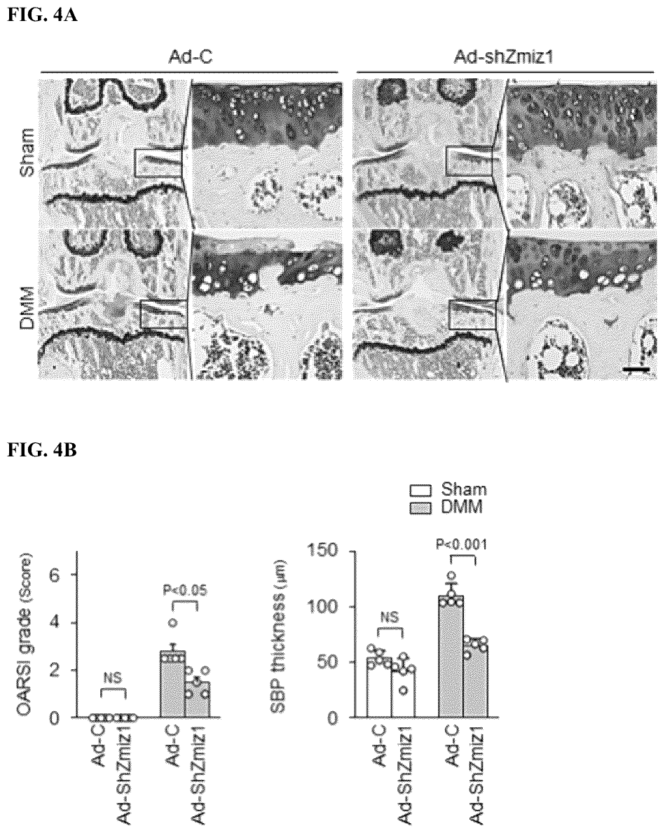

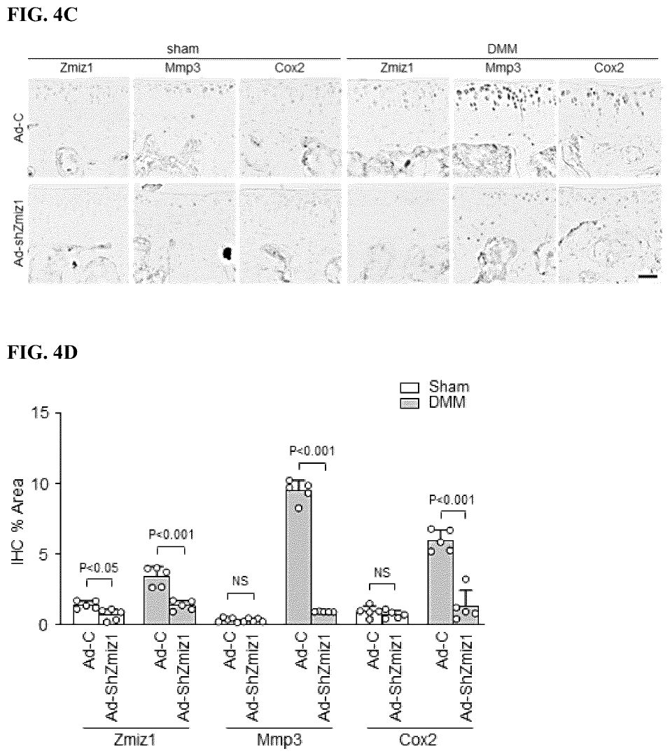

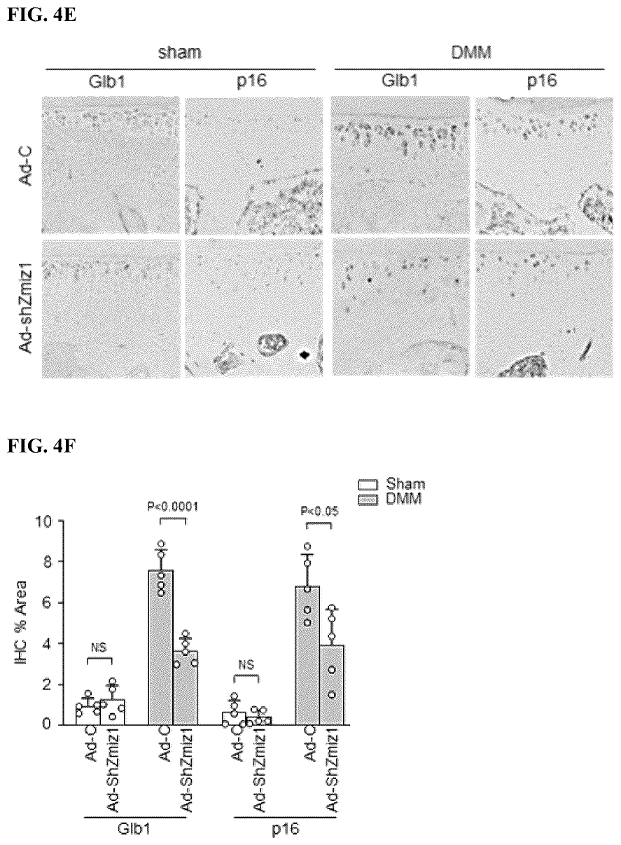

As a result, Safranin-O staining showed suppression of severe cartilage destruction and symptoms of osteoarthritis in the joint tissue of WT mice injected with Ad-shZmiz1 as shown in FIG. 4A, and at the same time, reduction was observed in the OARSI grade and SBP thickness as shown in FIG. 4B. When osteoarthritis occurred, it was shown in FIGS. 4C and D that cartilage destructing factors Mmp3 and Cox2 were reduced by suppressing expression of Zmiz1. In addition, it was shown in FIGS. 4E and 4F that the expression of senescent cell promoting factors Glb1 and p16 was reduced when Zmiz1 was not expressed upon occurrence of osteoarthritis.

### <Example 4> Identification of Transcription Factors Related to Zmiz1

45-pathway reporter (Qiagen, hilden, Germany) was used to overexpress adenovirus Zmiz1 in chondrocytes and identify transcription factors regulated by Zmiz1.

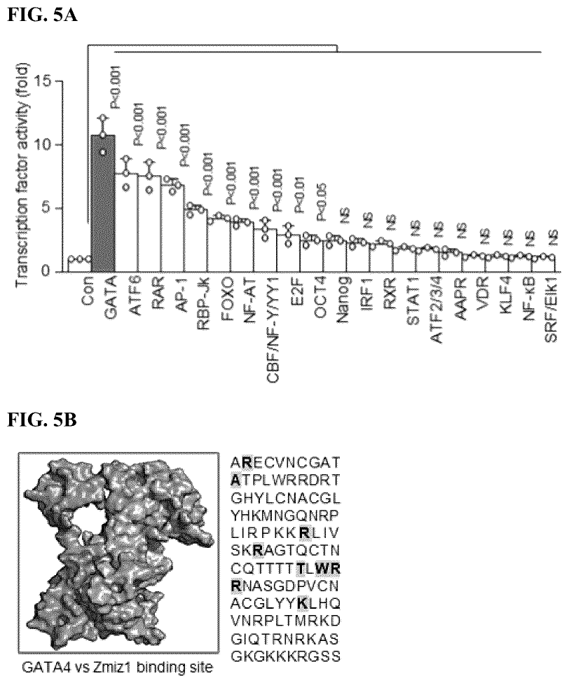

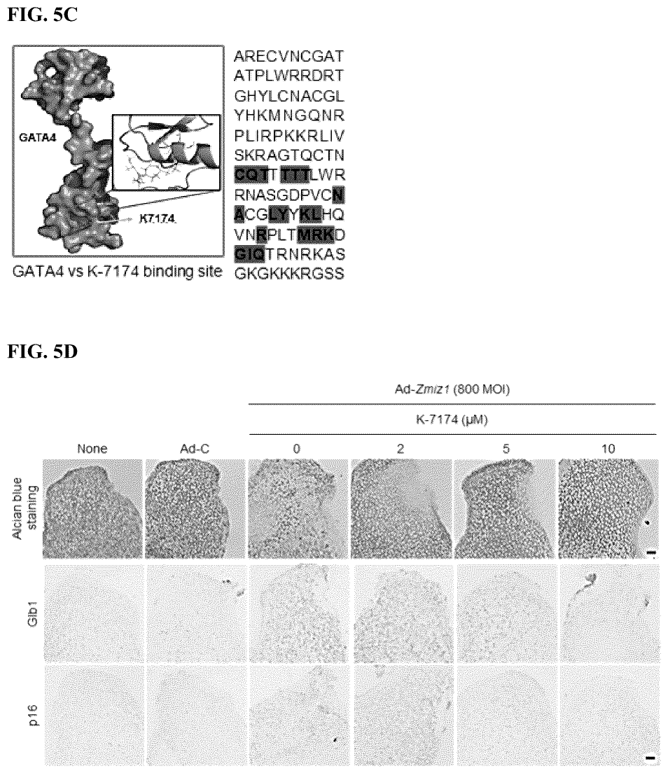

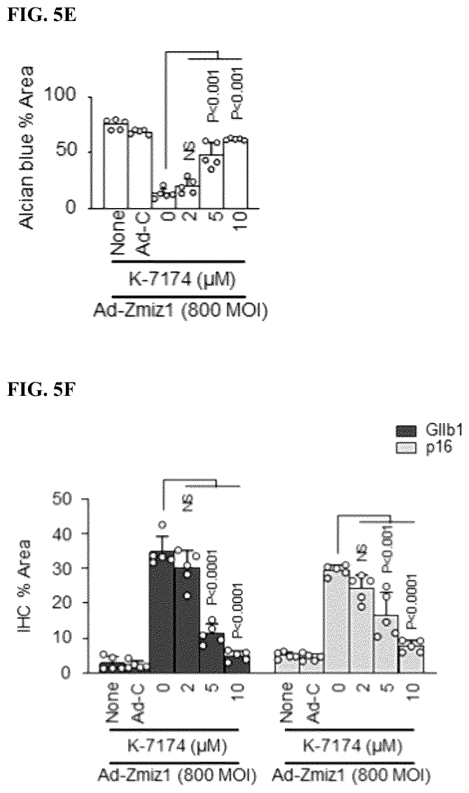

As a result, as shown in FIG. 5A, it was found that GATA was most regulated by Zmiz1.

### <Example 5> Identification of the Binding Between GATA and K-7174 and Binding Sites of Zmiz1 and K-7174 Using Discovery Studio

As a result of conducting molecular docking analysis using Discovery studio, as shown in FIG. 5B, it showed that the binding took place due to the binding between the GATA amino acid sequence and the Zmiz1 amino acid sequence, and at the same time, Zmiz1 and K-7174 may bind together as shown in FIG. 5C.

From the above results, it was found that K-7174 may inhibit the binding of Zmiz1 and GATA by binding to Zmiz1, thereby suppressing the expression of joint destructing factors and senescent cell promoting factors by Zmiz1.

### <Example 6> Identification of Suppression of Zmiz1-Mediated Senescent Cell Factor Expression and Promotion of ECM Secretion by K-7174

It was determined whether K-7174 is able to inhibit degradation of the extra cellular matrix (ECM) in Zmiz1-mediated joints and promotion of senescent cells. In order to determine whether K-7174 may suppress expression of decreasing sulfated proteoglycans and increasing senescent cell promoting factors (Glb1, p16) by infection with Ad-Zmiz1 while culturing mouse joints in a culture medium ex vivo to induce overexpression, K-7174 was treated to chondrocytes, wherein sulfated proteoglycan was observed by alcian blue staining and densitometry, and expression of senescent cell factors by immunohistochemistry and densitometry analysis.

As a result, it was found that sulfated proteoglycans reduced by Zmiz1 overexpression were increased in a K-7174 dose-dependent manner, as shown in FIG. 5D and FIG. 5E. At the same time, as shown in FIGS. 5D and 5F, it was found that the senescent cell promoting factors Glb1 and p16, which were increased by Zmiz1 overexpression, were reduced by K-7174.

### <Example 7> Identification of Inhibition in Osteoarthritis Induction and Promotion of Senescent Cell Elimination by K-7174

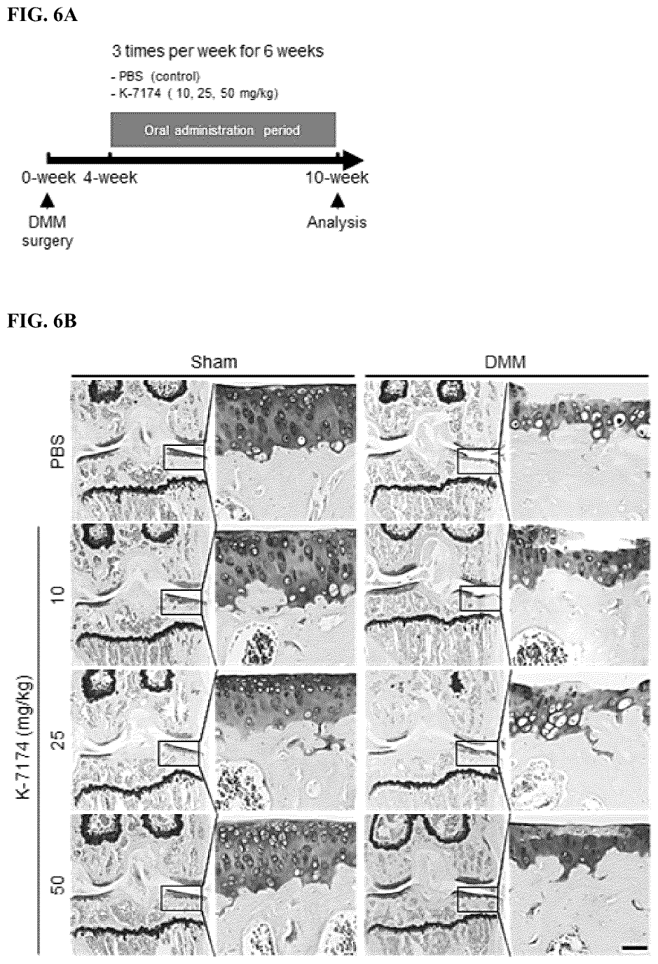

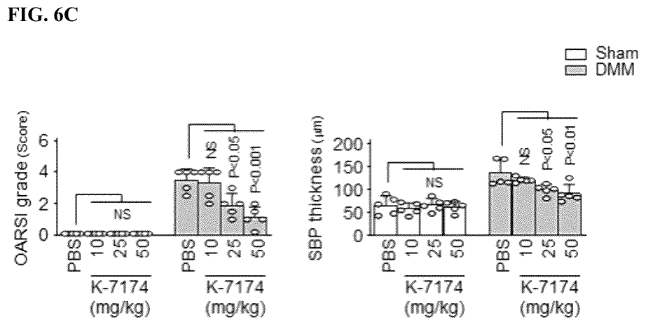

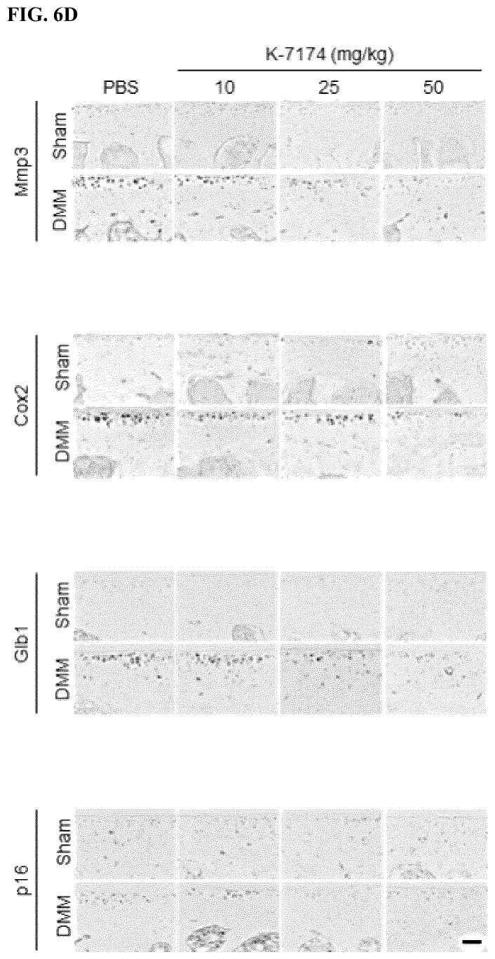

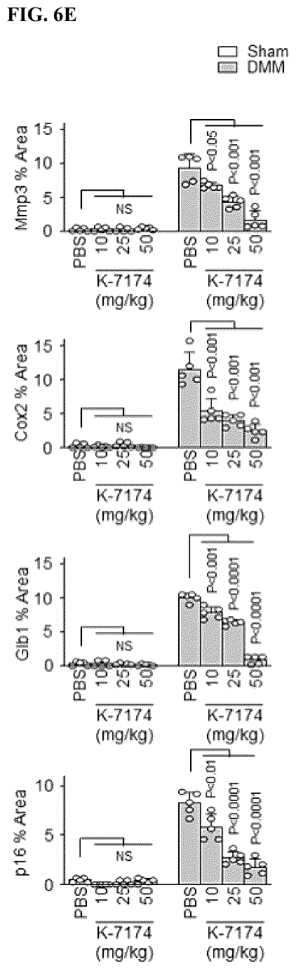

In an animal model with DMM-induced osteoarthritis, K-7174 (10, 25 and 50 mg/kg) was administered orally three times for 6 weeks in the same process as in FIG. 6A.

As a result, it was found that joint destruction induced by osteoarthritis was inhibited as shown in FIGS. 6B and 6C, and it was found by immunohistochemistry analysis that the expression of cartilage destructing factors (Mmp3, Cox2) and senescent cell promoting factors (Glb1, p16) was also reduced by K-7174 administration as shown in FIGS. 6D and 6E.

As specific parts of the present disclosure have been described in detail above, it is clear to those skilled in the art that these specific descriptions are merely preferred example embodiments and the scope of the present disclosure is not limited thereby. Thus, the substantial scope of the present disclosure will be defined by the attached claims and their equivalents.

1. A method of treating osteoarthritis, comprising:
administering a pharmaceutical composition comprising an expression or activity inhibitor of zinc finger MIZ domain-containing protein 1 (Zmiz1) as an active ingredient to a subject.

2. The method of claim 1, wherein the expression inhibitor of Zmiz1 is one or more selected from the group consisting of antisense nucleotides, siRNA, shRNA, microRNA, and ribozymes that specifically bind to mRNA of a Zmiz1 gene or a gene that promotes expression of Zmiz1.

3. The method of claim 1, wherein the activity inhibitor of Zmiz1 is one or more selected from the group consisting of compounds, peptides, peptidomimetics, proteins, aptamers, and antibodies that bind specifically to a protein that promotes Zmiz1 activity or a Zmiz1 protein.

4. A method of treating osteoarthritis, comprising:
administering a pharmaceutical composition comprising K-7174 as an active ingredient to a subject.

5. The method of claim 4, wherein the K-7174 binds to GATA whose expression is induced by Zmiz1.

6. The method of claim 4, wherein the K-7174 suppresses expression of MMP3 and COX2 induced by Zmiz1.

7. The method of claim 4, wherein the K-7174 suppresses expression of senescent cell promoting factors Glb1 or p16 which is increased by overexpression of Zmiz1.

8. The method of claim 4, wherein the pharmaceutical composition comprises the K-7174 in an amount of 0.1 to 10 parts by weight with respect to the total of 100 parts by weight of the composition.

9. A method of eliminating senescent cells, comprising:
administering a pharmaceutical composition comprising K-7174 as an active ingredient to a subject.
